(19) United States
(12) Patent Application Publication
(10) Pub. No.: US 2021/0286034 A1
(43) Pub. Date: Sep. 16, 2021

(54) MAGNETIC RESONANCE IMAGING DEVICE

(71) Applicant: Hitachi, Ltd., Chiyoda-ku, Tokyo (JP)

(72) Inventor: Hikaru HANADA, Tokyo (JP)

(73) Assignee: Hitachi, Ltd., Chiyoda-ku, Tokyo (JP)

(21) Appl. No.: 16/318,936

(22) PCT Filed: Jul. 12, 2017
(86) PCT No.: PCT/JP2017/025373

§ 371 Date: Jan. 18, 2019

(30) Foreign Application Priority Data
Jul. 28, 2016 (JP) 2016-148508

(51) Int. Cl. G01R 33/385

(52) U.S. Cl. CPC G01R 33/3854

(57) ABSTRACT

To reduce vibration resulting from applying a gradient magnetic field pulse. A gradient magnetic field coil is supported on a static magnetic field generating device. A control unit generates a gradient magnetic field pulse of a predetermined waveform in the gradient magnetic coil at predetermined timing, and executes a predetermined imaging pulse sequence including the gradient magnetic field pulse. The control unit includes a waveform determination unit determining a waveform of a gradient magnetic field pulse, and the waveform determination unit is configured to determine a waveform of the gradient magnetic field pulse so as to reduce a vibration transmissibility of a propagation path including a support for the gradient magnetic field coil and a table so as to prevent a force generated in the gradient magnetic field coil when a current is passed through the gradient magnetic field coil from being conveyed to the subject by way of the propagation path and fluctuation is caused in the subject's position.

### TECHNICAL FIELD

The present invention relates to a magnetic resonance imaging (hereafter, referred to as MRI) device that measures a nuclear magnetic resonance signal (hereafter, referred to as NMR signal) from hydrogen, phosphorus, or the like in a subject and performs imaging of a density distribution of a nucleus, a relaxation time distribution, or the like and in particular to a technology for suppressing vibration in the device caused by a gradient magnetic field pulse.

### BACKGROUND ART

The MRI device is a device that measures an NMR signal produced by a nuclear spin constituting a subject, especially, a human body tissue and scans a two-dimensional or three-dimensional image of the shape or functions of the head, abdomen, extremity, or the like of a subject. NMR signals are acquired as FID (Free Induction Decay) signals or echo signals. However, since almost all the NMR signals are acquired as an echo signal, NMR signals will be hereafter also referred to as echo signals. To pick up an image, a subject is placed in a static magnetic field and a high frequency magnetic field pulse is applied together with a slice selection gradient magnetic field pulse to selectively excite a specific region. Thereafter, a phase encoding gradient magnetic field pulse or a readout gradient magnetic field pulse is applied, thereby encoding the excited area and giving positional information. A measured echo signal is subjected to two-dimensional or three-dimensional Fourier transform and is thereby reconstructed into an image.

In measurement using such an MRI device, the shape or the timing of application and irradiation of a gradient magnetic field pulse and a high frequency magnetic field pulse is varied according to the purpose of the measurement. As a result, an image in which various tissues or the physiology of an organism is enhanced is obtained. One of such images is DWI (Diffusion Weighted Image). In scanning a diffusion weighted image, a gradient magnetic field pulse high in magnetic field strength called MPG (Motion Probing Gradient) is applied and the diffusive motion of water molecules is thereby reflected in the contrast of an image.

Meanwhile, PTL 1 discloses a technology for reducing sound produced when a gradient magnetic field coil generates a gradient magnetic field. In this technology, a gradient magnetic field coil is caused to generate a gradient magnetic field and a sound produced at that time is collected with a microphone and a relation between the frequency band of a gradient magnetic field waveform and a sound pressure level is measured. Then, a frequency band in which a sound pressure level becomes equal to or higher than a predetermined value is determined. That frequency band is removed from a gradient magnetic field waveform generated in an imaging pulse sequence and then the waveform is shaped. As a result, a sound pressure produced when a gradient magnetic field coil generates a gradient magnetic field is reduced.

### CITATION LIST

### Patent Literature

PTL 1: Japanese Patent Application Laid-Open No. 2015-231417

### SUMMARY OF INVENTION

### Technical Problem

As a result of an investigation, the present inventors found that: an MPG pulse applied when scanning a diffusion weighted image is relatively long in application time (the order of several to several tens of ms); therefore, a sound pressure level thereof is not high but a problem of a magnetic field vibration and the vibration of a subject's position being caused arises.

Magnetic field vibration is caused as follows: the shape and position of a gradient magnetic field coil are varied by Lorentz force exerted on the gradient magnetic field coil when a current is passed through the gradient magnetic field coil and a magnetic field distribution is thereby varied. When a magnetic field vibrates, a gradient magnetic field cannot be applied with an intended strength and this degrades the formability of an image. The vibration of a subject's position is caused as follows: positional fluctuation in a gradient magnetic field coil is propagated to the subject by way of a structure supporting the gradient magnetic field coil, for example, a static magnetic field generating device, a floor supporting the static magnetic field generating device, and a table placed on the floor.

A sound arising from a gradient magnetic field pulse is produced by a gradient magnetic field coil and propagates an imaging space and directly conveyed to a subject's ear. Meanwhile, vibration is conveyed through a path different from that of sound as mentioned above. When a subject vibrates, the motion of the subject due to the vibration is reflected in the contrast of an image and unwanted information is mixed. This causes degradation in image quality. In addition, vibration conveyed to the subject can give the subject discomfort. Magnetic field vibration and the vibration of a subject's position depend on not only the magnitude of a gradient magnetic field pulse but also a supporting structure for the gradient magnetic field coil and a mechanism for fixing the MRI device on a floor.

PTL 1 proposes a technology for reducing a sound produced by a gradient magnetic field coil.

However, the vibration of a subject's position involves two different elements, magnetic field and structure, as mentioned above and these elements are different from each other in the mechanism of image quality degradation and influence on the subject. Therefore, further contrivance is required to suppress degradation in image quality and discomfort to a subject by the technology in PTL 1.

The present invention has been made in consideration of the abovementioned problem and it is an object of the present invention to provide a technology for suppressing vibration due to the application of a gradient magnetic field pulse.

### Solution to Problem

To solve the abovementioned problem, an MRI device in accordance with the present invention includes: static magnetic field generating device supplying a static magnetic field to an imaging space in which a subject is placed; a table for placing a subject in the imaging space; a gradient magnetic field coil applying a gradient magnetic field pulse to the imaging space; a gradient magnetic field power supply supplying a current of a predetermined waveform to the gradient magnetic field coil to generate a gradient magnetic field pulse; a support supporting the gradient magnetic field coil; and a control unit that controls and causes the gradient magnetic field power supply to apply a gradient magnetic field pulse of a predetermined waveform to the imaging space at predetermined timing and execute a predetermined imaging pulse sequence including the gradient magnetic field pulse.

The control unit includes a waveform determination unit determining a waveform of a gradient magnetic field pulse. The waveform determination unit determines a waveform of a gradient magnetic field pulse so as to reduce a vibration transmissibility of a propagation path including the support for the gradient magnetic field coil and the table. This prevents a force generated in the gradient magnetic field coil when a current is passed through the gradient magnetic field coil from being conveyed to the subject by way of the propagation path, causing fluctuation in the subject's position.

### Advantageous Effects of Invention

According to the present invention, it is possible to reduce vibration due to the application of a gradient magnetic field pulse.

### BRIEF DESCRIPTION OF DRAWINGS

FIG. 1 is a block diagram illustrating an overall configuration of an MRI device in a first embodiment.

FIG. 2 is a drawing illustrating an example of a pulse sequence for DWI.

FIG. 3 is a block diagram illustrating a configuration of a waveform determination unit in the first embodiment.

FIG. 4 is a flowchart illustrating operation of a window function computation processing unit of a waveform determination unit in the first embodiment.

FIG. 5 is a graph indicating an example of a distribution of vibration strength for each vibration frequency of an MRI device observed when a vibration source is a gradient magnetic field coil.

FIG. 6 is a drawing illustrating an example screen for accepting a degree of vibration reduction from a user in the first embodiment.

FIG. 7 is a graph indicating an example of a window function in the first embodiment.

FIG. 8 is a flowchart illustrating operation of a low vibration MPG pulse computation processing unit in the first embodiment.

FIGS. 9(a) to 9(d) are graphs indicating variation in a waveform of a gradient magnetic field pulse caused by processing of a low vibration MPG pulse computation processing unit in the first embodiment.

FIG. 10(a) is a graph indicating a frequency spectrum of a gradient magnetic field pulse before processing by a low vibration MPG pulse computation processing unit in the first embodiment; FIG. 10(b) is a graph indicating a frequency spectrum after the application of a window function by the low vibration MPG pulse computation processing unit; and FIG. 10(c) is a graph indicating a frequency spectrum of a gradient magnetic field pulse after processing by the low vibration MPG pulse computation processing unit.

FIG. 11 is a graph indicating the graphs in FIG. 10(a) to FIG. 10(c) consolidated and shown in a partly enlarged manner.

FIGS. 12(a) to 12(c) are graphs indicating an example of a distribution of vibration strength for each vibration frequency of an MRI device (static magnetic field generating device) observed when each of the X, Y, and Z-axis coils of a gradient magnetic field coil is a vibration source.

FIG. 13 is a flowchart illustrating operation of a low vibration MPG pulse computation processing unit in a second embodiment.

FIG. 14 is a drawing illustrating an example of a pulse sequence for DWI involving a crusher.

### DESCRIPTION OF EMBODIMENTS

Hereafter, a detailed description will be given to an MRI device in embodiments of the present invention with reference to the accompanying drawings. In all the drawings referred to in relation to a description of each embodiment, items having an identical function will be marked with identical reference signs and a repetitive description thereof will be omitted.

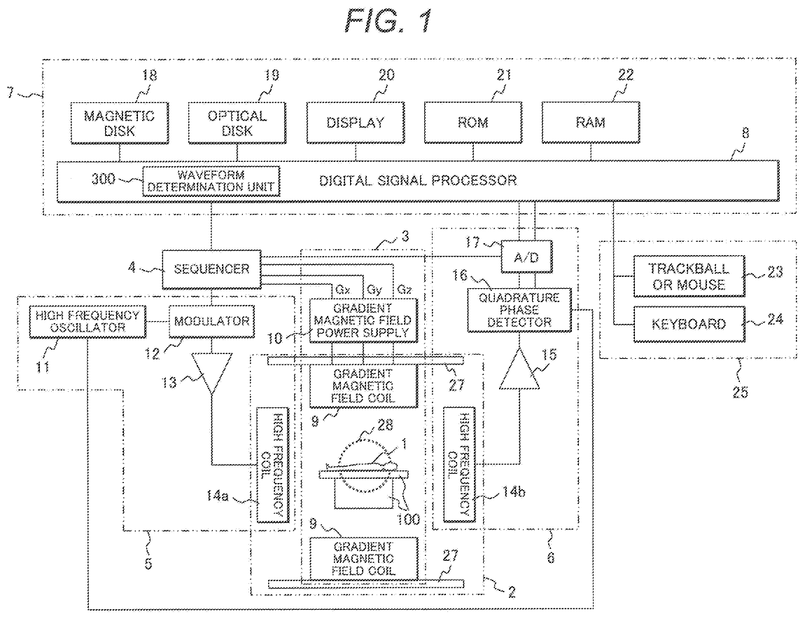

A description will be given to an example of an overall configuration of an MRI device in an embodiment with reference to FIG. 1.

FIG. 1 is a block diagram illustrating an overall configuration of an MRI device in an embodiment. This MRI device utilizes an NMR phenomenon to obtain a tomographic image of a subject. As shown in FIG. 1, the MRI device includes a static magnetic field generation system 2, a table 100, a gradient magnetic field generation system 3, a transmission system 5, a reception system 6, a signal processing system 7, and a sequencer 4.

The sequencer 4 is a control means for repetitively radiating and applying a high frequency magnetic field pulse and a gradient magnetic field pulse at predetermined timing (imaging pulse sequence). The sequencer 4 operates under the control of a digital signal processor (control unit) 8 located in the signal processing system 7. The sequencer sends various instructions to the transmission system 5, the gradient magnetic field generation system 3, and the reception system 6 and causes them to execute an imaging pulse sequence to collect data required for reconstructing a tomographic image of a subject 1.

The static magnetic field generation system 2 includes a static magnetic field generating device 27 disposed around an imaging space 28 in which the subject 1 is placed and generates a uniform static magnetic field in the imaging space 28. In cases where the static magnetic field generation system 2 is of a vertical magnetic field type, the orientation of the static magnetic field is orthogonal to the body axis of the subject 1 and a pair of the static magnetic field generating devices 27 is vertically disposed opposite to each other with the subject 1 in between. Meanwhile, in cases where the static magnetic field generation system 2 is of a horizontal magnetic field type, the orientation of the static magnetic field is identical with the direction of the body axis of the subject 1 and the static magnetic field generating device 27 is so shaped as to surround the body axis of the subject 1. The static magnetic field generating device 27 may be any of a permanent magnet type, a normal conduction type, and a superconductivity type.

The table 100 receives the subject 1 and places the subject 1 in the imaging space 28. In this example, the table 100 is so provided as to be supported by a floor surface on which the MRI device is installed but the table 100 may be partly or wholly supported by any other configuration element, such as the static magnetic field generating device 27.

The gradient magnetic field generation system 3 includes: a gradient magnetic field coil 9 wound in the directions of three axes, X, Y, and Z, which are a coordinate system (coordinate system at rest) of the MRI device; and a gradient magnetic field power supply 10 supplying a current to each gradient magnetic field coil 9 to drive it. The gradient magnetic field power supply 10 for each coil supplies a current of a predetermined pulse waveform to each gradient magnetic field coil 9 in accordance with an instruction received from the sequencer 4 and applies a gradient electromagnetic pulse in the directions of the three axes, X, Y, and Z.

For example, in picking up an image, a slice direction gradient magnetic field pulse Gs is applied to a direction orthogonal to a slice surface (surface to be imaged) to set a slice surface for the subject 1. Then, a phase encoding direction gradient magnetic field pulse Gp and a frequency encoding direction gradient magnetic field pulse Gr are applied to the remaining two directions orthogonal to that slice surface and further orthogonal to each other. Positional information for the respective directions is encoded into an echo signal.

The transmission system 5 irradiates the subject 1 with a high frequency magnetic field pulse and includes a high frequency oscillator 11, a modulator 12, a high frequency amplifier 13, and a transmitting high frequency coil (transmission coil) 14a. Using these configuration elements, the transmission system 5 irradiates the subject 1 with a high frequency magnetic field pulse causing a nuclear spin of an atom constituting a bio-tissue of the subject 1 to cause nuclear magnetic resonance. A more specific description will be given. The high frequency oscillator 11 outputs a high frequency signal and the modulator 12 amplitude modulates the high frequency electrical signal at timing in accordance with an instruction received from the sequencer 4. This amplitude modulated high frequency electrical signal is amplified by the high frequency amplifier 13 and is then supplied to the high frequency coil 14a. As a result, the subject 1 is irradiated with a high frequency magnetic field pulse from the high frequency coil 14a disposed in proximity to the subject 1.

The reception system 6 includes a receiving high frequency coil (reception coil) 14b, a signal amplifier 15, a quadrature phase detector 16, and an A/D converter 17. Using these configuration elements, the reception system 6 detects an echo signal (NMR signal) emitted by nuclear magnetic resonance of a nuclear spin constituting a bio-tissue of the subject 1. In other words, irradiated with a high frequency magnetic field pulse from the transmitting high frequency coil 14a, the subject 1 is excited and emits an NMR signal as a response signal. The high frequency coil 14b disposed in proximity to the subject 1 detects the NMR signal. The NMR signal is amplified at the signal amplifier 15 and is then divided into signals in two orthogonal systems by the quadrature phase detector 16 at timing in accordance with an instruction from the sequencer 4. Each signal is converted into a digital signal at the A/D converter 17 and sent to the signal processing system 7.

The signal processing system 7 includes a digital signal processor 8, an external storage such as an optical disk 19 and a magnetic disk 18, a display 20 comprised of CRT or the like, ROM 21, and RAM 22 and processes varied data, displays and stores a result of processing, and performs other like operations. Receiving a digital signal from the reception system 6, the digital signal processor 8 performs signal processing, image reconstruction, or the like and thereby reconstructs a tomographic image of the subject 1 and displays the image on the display 20 and further records the image on the magnetic disk 18 and the like as external storages.

An operating unit 25 is used for a user to input varied control information for the MRI device and control information for processing performed at the signal processing system 7 and includes a trackball or a mouse 23 and a keyboard 24. The operating unit 25 is placed in proximity to the display 20 and an operator interactively controls varied processing of the MRI device via the operating unit 25 while watching the display 20.

In FIG. 1, the transmitting high frequency coil 14a and the gradient magnetic field coil 9 are so shaped as to be opposed to each other with the subject 1 in between in cases where the static magnetic field generation system 2 is of a vertical magnetic field type and are so shaped as to surround the body axis of the subject 1 in cases where the static magnetic field generation system 2 is of a horizontal magnetic field type. In this example, the high frequency coil 14a and the gradient magnetic field coil 9 are fixed and supported on the imaging space 28-side wall face of the static magnetic field generating device 27.

Meanwhile, the receiving high frequency coil 14b is so disposed as to be opposed to or surround the subject 1. The gradient magnetic field coil 9 need not be so structured as to be fixed on a wall face of the static magnetic field generating device 27 and may be separately provided with a support and may be so structured as to be supported directly on a floor surface on which the MRI device is installed.

One of nuclides to be imaged with an MRI device presently widespread in the field of medicine is hydrogen nucleus (proton) as a major constituent substance of each subject. The shape or functions of the head, abdomen, extremity, or the like of a human are scanned as a two-dimensional or three-dimensional image by imaging information on a spatial distribution of proton density or a spatial distribution of a relaxation time of an excited state.

A description will be given to an example of an imaging pulse sequence (hereafter, simply referred to as pulse sequence) executed by the sequencer 4.

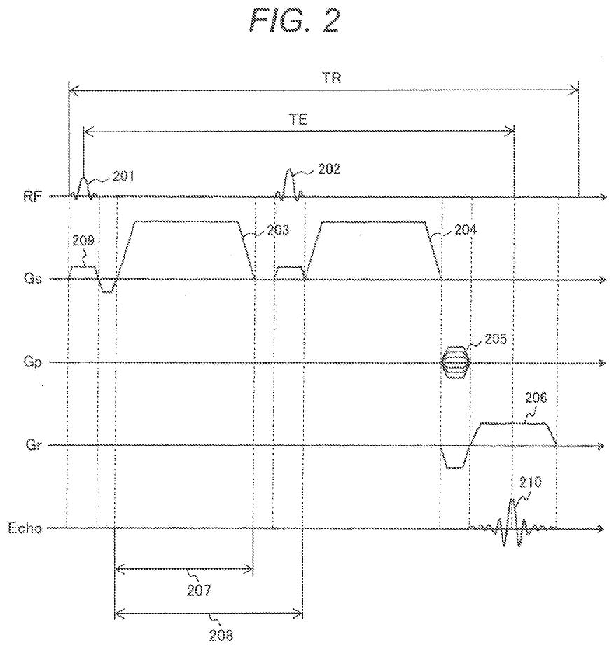

FIG. 2 shows an example of a pulse sequence for DWI. In the pulse sequence for DWI, first, while a slice selection gradient magnetic field pulse (Gs) 209 is applied, a high frequency magnetic field pulse 201 is radiated to excite a spin in a specific slice position. Thereafter, a first MPG pulse 203 is applied. In the example in FIG. 2, an MPG pulse is applied to the axis of the slice selection gradient magnetic field pulse 209 but may be applied to any other axis or a plurality of axes.

After the application of the first MPG pulse 203, a high frequency magnetic field pulse 202, called 180°-RF pulse, inverting a spin phase is radiated and a second MPG pulse 204 is then applied. The area (gradient magnetic field strength×application time) of the first MPG pulse 203 and the area of the second MPG pulse 204 are equal to each other. After the application of the phase encoding gradient magnetic field pulse (Gp) 205, while a frequency encoding gradient magnetic field pulse (Gf) 206 is applied, an echo signal 210 is received. This operation (repetition time TR) is repeated by a predetermined number of times while varying the gradient magnetic field strength of the phase encoding gradient magnetic field pulse 205. From data on echo signals 210 obtained as a result, a cross-sectional image of a selected slice is reconstructed.

In a pulse sequence for DWI, for a spin whose spatial position within a selected slice of a subject 1, the phase thereof varied by the first MPG pulse 203 is returned to the original phase by the second MPG pulse 204. Meanwhile, for a spin whose spatial position is shifted (diffused) in the direction of MPG pulse application, the phase thereof varied by the first MPG pulse 203 is not completely returned to the original phase by the second MPG pulse 204. For this reason, a phase difference is produced between that spin and the surrounding spins and macroscopically an echo signal is attenuated. Therefore, in cases where in a slice of a subject 1, there is a location (spin) shifted (diffused) in the direction of application of the first MPG pulse 203, that location appears as a low signal region in a reconstructed image and the direction of shift (diffusion) can be highlighted.

Consequently, respective directions of shift (diffusion) can be grasped by varying the direction of application of the first MPG pulse 203 to obtain a plurality of reconstructed images.

In the abovementioned pulse sequence, instead of use of the 180°-RF pulse 202, the polarity of the second MPG pulse 204 may be set opposite to the polarity of the first MPG pulse 203. Also, in this case, the same effect is obtained.

The abovementioned first and second MPG pulses 203, 204 are high in gradient magnetic field strength and relatively long in application time and give vibration to a subject. To cope with this, an MRI device in embodiments of the present invention is provided with a waveform determination unit determining a waveform of a gradient magnetic field pulse for vibration reduction. Hereafter, a description will be given to a configuration of a waveform determination unit.

### First Embodiment

As shown in FIG. 1, an MRI device in the first embodiment is provided in the digital signal processor (control unit) 8 causing the sequencer 4 to perform a predetermined imaging pulse sequence with a waveform determination unit 300 determining a waveform of a gradient magnetic field pulse. The waveform determination unit 300 determines a waveform of a gradient magnetic field pulse so as to reduce a vibration transmissibility of a propagation path including the support for the gradient magnetic field coil 9 and the table 100. Thus, a force generated in the gradient magnetic field coil 9 when a current is passed through the gradient magnetic field coil 9 is prevented from being conveyed to the subject 1 by way of the propagation path, causing fluctuation in the subject 1's position. More specifically, for example, the waveform determination unit 300 uses a relation between the frequency of a current supplied to the gradient magnetic field coil 9 and the magnitude of resultant vibration (vibration strength) in the table 100, obtained in advance to select a frequency component with which the magnitude of vibration at the relevant frequency is equal to or lower than a predetermined value. A waveform of a gradient magnetic field pulse is determined with the selected frequency component.

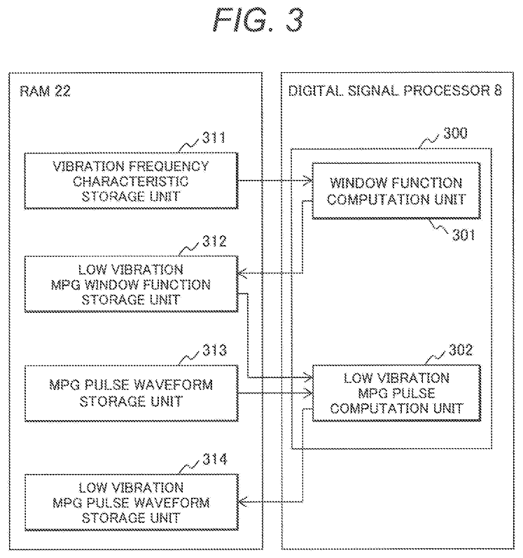

A description will be given to a configuration of the waveform determination unit 300 with reference to the block diagram in FIG. 3. As shown in FIG. 3, the waveform determination unit 300 includes a window function computation unit 301 and a low vibration MPG pulse computation unit 302.

Meanwhile, the RAM 22 includes a vibration frequency characteristic storage unit 311, a low vibration MPG window function storage unit 312, an MPG pulse waveform storage unit 313, and a low vibration MPG pulse waveform storage unit 314. The vibration frequency characteristic storage unit 311 has a vibration frequency characteristic stored in advance which characteristic indicates a relation between the frequency of a current supplied to the gradient magnetic field coil 9 and the magnitude of resultant vibration in the table 100 obtained beforehand. The MPG pulse waveform storage unit 313 holds a waveform of an MPG pulse used for a pulse sequence for DWI on an imaging condition-by-imaging condition basis, for example, by a function representing a waveform.

The window function computation unit 301 performs processing to obtain a window function for selecting a frequency component with which the magnitude of vibration at the relevant frequency is equal to or lower than a predetermined value. An obtained window function is stored in the low vibration MPG window function storage unit 312 of the RAM 22. Processing for the window function computation unit 301 to obtain a window function only has to be performed once as initialization after the manufacture of the MRI device and it is especially desirable to perform the processing after engineering work for installing the MRI device in place. This is because a vibration frequency characteristic is varied depending on a mechanism for fixing the MRI device on an installation site or a structure of the installation site. Since this processing to obtain a window function only has to be performed once, the digital signal processor 8 need not always be provided with the window function computation unit 301 and a window function computed with an external processor may be stored in the low vibration MPG window function storage unit 312.

The low vibration MPG pulse computation unit 302 uses a window function computed by the window function computation unit 301 to correct a waveform of an MPG pulse in a pulse sequence for DWI generated based on various measurement conditions into a low vibration waveform.

The window function computation unit 301 and the low vibration MPG pulse computation unit 302 of the waveform determination unit 300 may be implemented by software or may be implemented by hardware. In cases where the waveform determination unit 300 is implemented by software, the functions of the window function computation unit 301 and the low vibration MPG pulse computation unit 302 are implemented by a processing unit, such as CPU, built in the digital signal processor 8 reading and executing a predetermined program stored in memory in advance. In cases where some or all of the waveform determination unit 300 is implemented by hardware, some or all of the functions of the window function computation unit 301 and the low vibration MPG pulse computation unit 302 are implemented by hardware including a custom IC such as ASIC (Application Specific Integrated Circuit) and a programmable IC such as FPGA (Field-Programmable Gate Array).

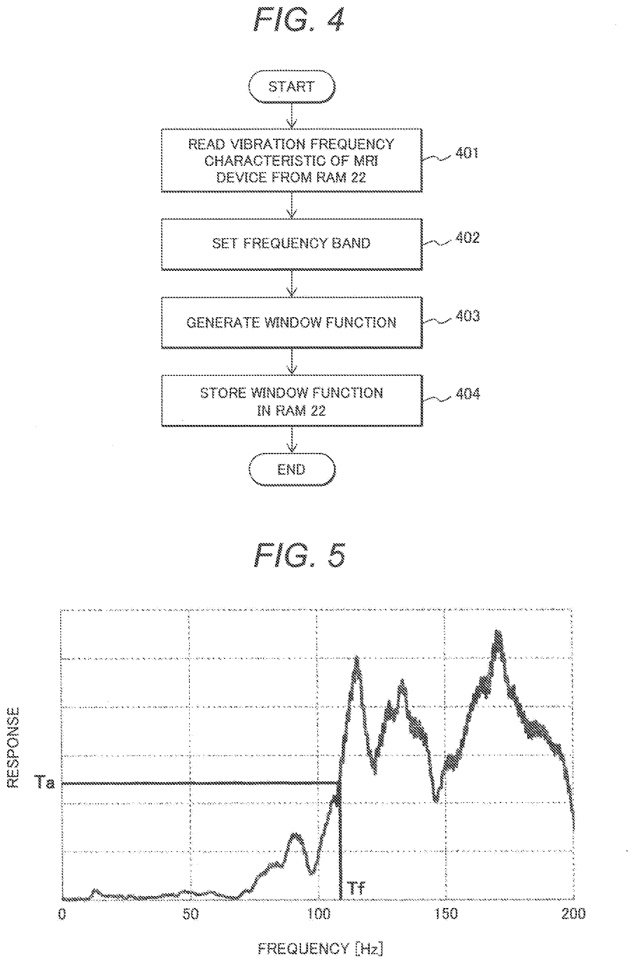

A description will be given to a flow of processing of the window function computation unit 301 with reference to the flowchart in FIG. 4. At Step 401 in FIG. 4, the window function computation unit 301 reads a vibration frequency characteristic of the MRI device from the vibration frequency characteristic storage unit 311 of the RAM 22.

A vibration frequency characteristic is obtained by varying the frequency of a waveform of a current passed through the gradient magnetic field coil 9 as a vibration source and further recording the magnitude of vibration (acceleration in this example) for each frequency with a vibrometer attached to the table 100 on which the subject 1 is placed. As mentioned above, there are two elements in vibration: magnetic field vibration and the vibration in a subject's position. A vibration frequency characteristic containing these two elements is obtained through measurement with the vibrometer attached to the table 100. This is because variation in the shape and position of the gradient magnetic field coil 9 vibrating a magnetic field is conveyed to the subject and becomes vibration varying the position thereof and thus the two elements simultaneously occur. The obtained vibration frequency characteristic is stored in the vibration frequency characteristic storage unit 311. A vibration frequency characteristic may be recorded for each direction of vibration (for example, X, Y, or Z direction), may be recorded for each vibration source (for example, the X, Y, or Z-axis coil constituting the gradient magnetic field coil 9), or may be recorded for each vibration frequency characteristic measurement position.

FIG. 5 indicates an example of a vibration frequency characteristic. In this embodiment, the vibration frequency characteristic VFC(f) is expressed by Expression (1) and stored in the vibration frequency characteristic storage unit 311.

[Expression 1]

VFC(f)=Max[ACC(Source,Axis,f)] (1)

In Expression (1), f denotes frequency; Max[ ] is a function indicating the maximum value within [ ]; ACC( ) is a data row of acceleration measured for each direction of shake source, each direction of acceleration, and each frequency; Source indicates the direction (X, Y, Z) of a shake source; and Axis indicates the direction (X, Y, Z) of acceleration. That is, a function indicating the maximum acceleration for each direction of shake source, each direction of acceleration, and each frequency is the vibration frequency characteristic VFC(f).

This embodiment is so configured that a vibration frequency characteristic measured beforehand is stored in the vibration frequency characteristic storage unit 311 in advance. Instead, the window function computation unit 301 may conduct processing of measuring a vibration frequency characteristic. For example, at Step 401, the window function computation unit 301 may instruct the sequencer 4 to cause the gradient magnetic field coil 9 to supply a current while varying a frequency. Then, the magnitude of vibration (acceleration in this example) is recorded for each frequency with the vibrometer attached to the table 100 and a vibration frequency characteristic is thereby measured and stored in the vibration frequency characteristic storage unit 311.

At Step 402, subsequently, the window function computation unit 301 sets a frequency band for making an MPG pulse a low vibration pulse from the vibration frequency characteristic read at Step 401. For example, in a vibration frequency characteristic indicated as in FIG. 5, a frequency high in response (vibration) level is avoided and only a frequency low in response level is used to configure a waveform of an MPG pulse. Thus, the sensitivity of response to a vibration source (gradient magnetic field coil 9) is lowered and vibration is reduced.

As shown in FIG. 5, the vibration level of a vibration frequency characteristic is high in a predetermined band (120 to 200 Hz in the example in FIG. 5) and is low in bands of lower frequencies and higher frequencies. However, composing a gradient magnetic field pulse of a high frequency component generally raises a noise level and is undesirable. For this reason, the window function computation unit 301 selects a frequency component low in response gain (vibration level to the value of current supplied to the gradient magnetic field coil 9) in a frequency band of low frequencies. Specifically, the window function computation unit 301 determines an allowable maximum response gain Ta and sets the lowest frequency Tf among the frequencies delivering a response gain exceeding Ta as the maximum frequency constituting an MPG pulse. In the example of the vibration frequency characteristic in FIG. 5, the maximum frequency Tf is 105 Hz. As the allowable maximum response gain Ta is lowered, a vibration reduction ratio becomes higher and as the allowable maximum response gain Ta is set higher, a vibration reduction ratio becomes lower.

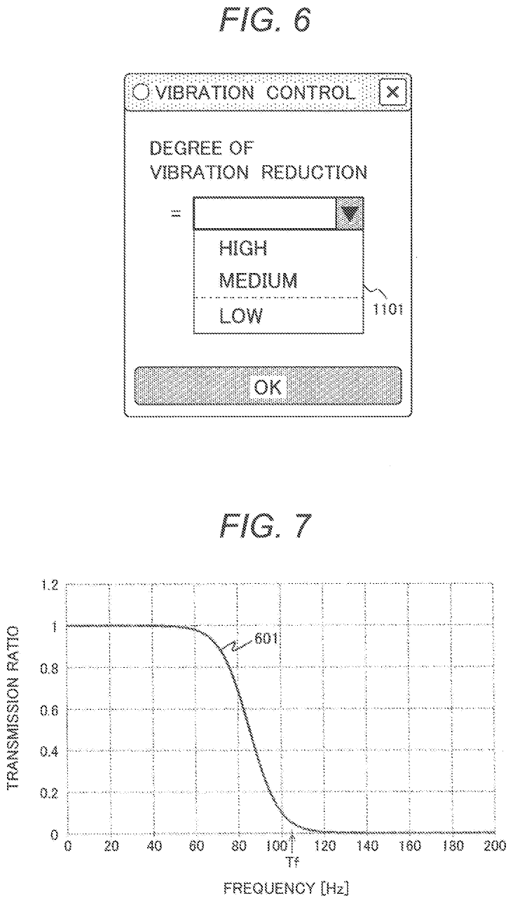

An allowable maximum response gain Ta may be uniquely defined using a predetermined value. Alternatively, values different in vibration reduction ratio may be prepared in advance and a value selected by a user from among them may be used. For example, the window function computation unit 301 displays such a UI (User Interface) as shown in FIG. 6 on the display 20 and accepts a selection of a degree of vibration reduction (reduction ratio) from a user operating the MRI device via the operating unit 25.

In the case of the example in FIG. 6, choices of degrees of reduction indicated as “High,” “Medium,” and “Low” are respectively associated with different allowable maximum response gains Ta beforehand. When a user selects a desired degree of vibration reduction from among them via the operating unit 25, the window function computation unit 301 establishes an allowable maximum response gain Ta corresponding to the selected degree of reduction. For example, for Medium, a default value is taken as the value of Ta, for High, a lower value is set for Ta, and for Low, a higher value is set for Ta.

At Step 403, the window function computation unit 301 executes processing to generate a low vibration MPG pulse using a frequency band equal to or lower than the allowable maximum frequency Tf. That is, the window function computation unit generates a window function that allows frequency bands of frequencies equal to or lower than the allowable maximum frequency Tf to transmit and blocks frequency bands of frequencies higher than the allowable maximum frequency Tf. Using this window function, frequency components constituting an MPG pulse are limited to frequency components of low vibration as described later and the MPG pulse is thereby generated from only frequency components low in vibration level.

Any window function can be used as long as frequency bands are limited as desired by the function but it is desirable to use a function with which a side lobe is less prone to be produced in an MPG pulse generated by limiting frequency bands. A trapezoidal wave is used for typical MPG pulses but when a rectangular window function is applied to a frequency component of a trapezoidal wave to limit frequency bands, a side lobe is produced in an MPG pulse and the application time of the MPG pulse is lengthened. In this example, consequently, a Fermi distribution function is used as a window function less prone to produce a side lobe.

The Fermi distribution function is defined by Expression (2).

$$\begin{matrix}[ Expression 2 ] & \\ W ( f ) = \frac{1}{1 + exp ( β ( f - μ ) )} & ( 2 )\end{matrix}$$

In Expression (2), f denotes frequency; β is a parameter for adjusting the steepness of a border between frequency bands to be blocked and frequency bands not to be blocked; and p is a parameter adjusting a bandwidth. For example, when 0.15 is taken for β and 5% is taken for transmissibility (transmission ratio) at the allowable maximum frequency Tf(105 Hz) established at Step 402, p is approximately 85.4. FIG. 7 is obtained by plotting W(f) as window function 601 expressed by Expression (2) with these values.

With the window function 601 in FIG. 7, the transmission ratio is high in low frequency bands equal to or lower than the allowable maximum frequency Tf(105 Hz) and especially, the transmission ratio in low frequency bands equal to or lower than a frequency of 50 Hz is approximately 100%. The transmission ratio at the allowable maximum frequency Tf is 5% or so and the transmission ratio in frequency bands of not less than 120 Hz higher than the frequency Tf is approximately zero. Within the frequency range from 50 Hz to 120 Hz, the transmission ratio is inclined and is gently varied.

At Step 404, the window function computation unit 301 stores the window function 601 generated at Step 403 in the low vibration MPG window function storage unit 312 of the RAM 22.

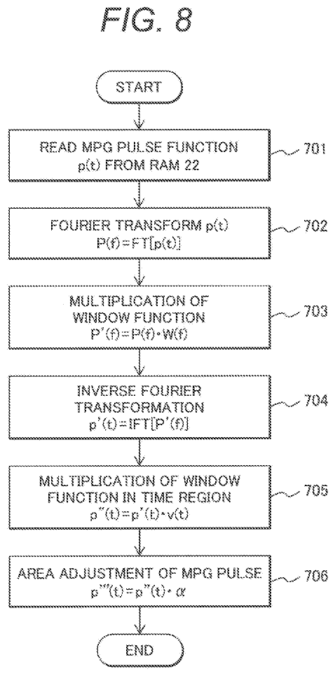

Description will be given to processing performed by the low vibration MPG pulse computation unit 302 with reference to the flowchart in FIG. 8. At Step 701, the low vibration MPG pulse computation unit 302 reads a waveform of an MPG pulse used in a pulse sequence for DWI from the MPG pulse waveform storage unit 313 of the RAM 22. A waveform of an MPG pulse is represented, for example, by a function and is stored beforehand in the MPG pulse waveform storage unit 313 on an imaging condition-by-imaging condition basis. The low vibration MPG pulse computation unit 302 reads a function indicating a waveform of an MPG pulse corresponding to an imaging condition for a pulse sequence for DWI specified by a user.

A waveform of an MPG pulse is lengthened in application time thereof as the result of frequency bands being limited at the subsequent Steps 702 to 706. At this point of time, consequently, the low vibration MPG pulse computation unit 302 performs processing to shorten the application time of the MPG pulse as much as possible.

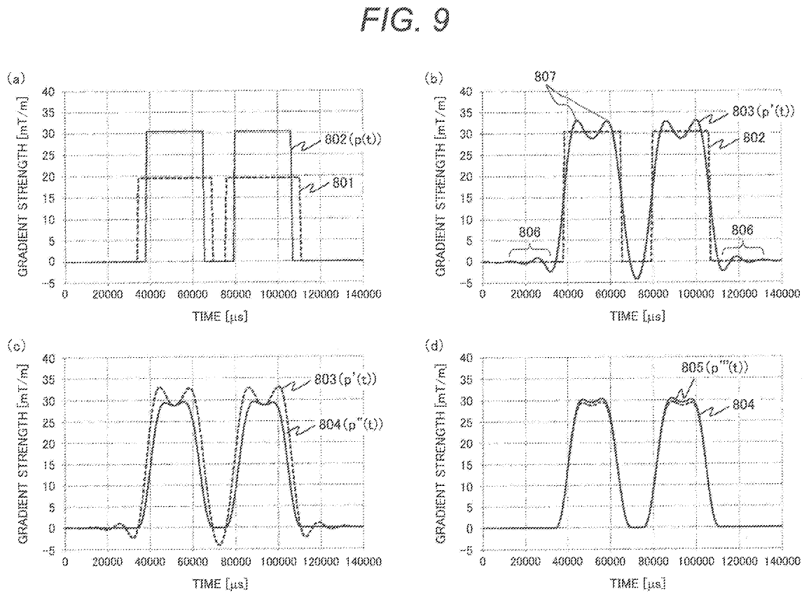

A specific description will be given. As indicated in FIG. 9(a), an MPG pulse waveform is deformed so that the maximum gradient magnetic field strength of an MPG pulse 801 read from the MPG pulse waveform storage unit 313 agrees with the maximum gradient magnetic field strength that can be applied by the MRI device. Further, the application time of the deformed MPG pulse 802 is shortened so that b-factor (=γ²G²δ² (Δ−δ/3)) is equal between when the deformed MPG pulse is used as the first and second MPG pulses 203, 204 in the pulse sequence for DWI in FIG. 2 and when the MPG pulse 801 before the deformation is used.

Here, γ denotes a gyromagnetic ratio; G denotes gradient magnetic field strength; δ is an MPG application time 207 (Refer to FIG. 2); and Δ denotes an MPG application interval 208 (Refer to FIG. 2). Instead of computing b-factor, the application time may be shortened so that the area (=gradient magnetic field strength×application time) of the MPG pulse 802 becomes equal to the area of the MPG pulse 801. As a result, as indicated in FIG. 9(a), the application time of the deformed MPG pulse 802 becomes shorter than the application time of the read MPG pulse and it is possible to apply the two MPG pulses with the respective shortest application times. A function representing a waveform of the changed MPG pulse 802 is expressed by p(t).

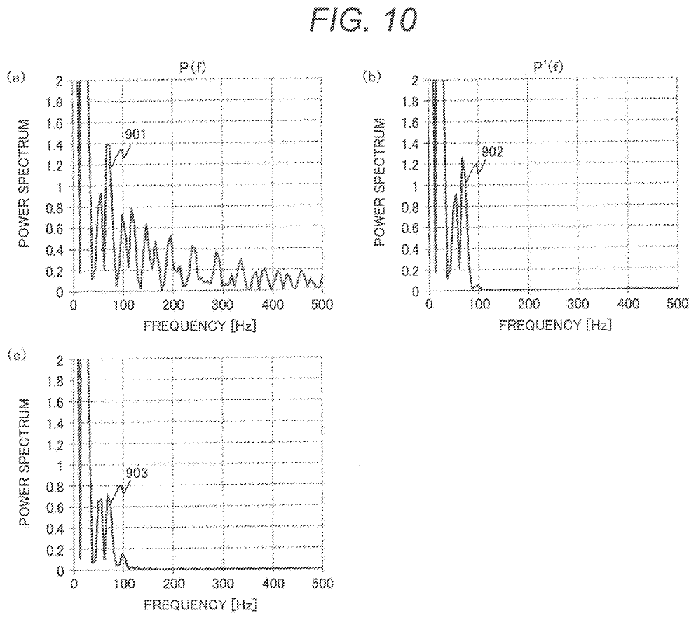

At Step 702, the low vibration MPG pulse computation unit 302 performs Fourier transform on the function p(t) to obtain a function P(f) indicating a frequency spectrum. FIG. 10(a) indicates an example of frequency spectrum 901 as the function P(f). Like the frequency spectrum 901 in FIG. 10(a), frequencies are distributed over a wide band.

At Step 703, the low vibration MPG pulse computation unit 302 reads a window function W(f) stored in the low vibration MPG window function storage unit 312 of the RAM 22 at Step 404, and acquires a function P′(f) by multiplying a function P(f) of a frequency spectrum by the window function W(f). Accordingly, as shown in FIG. 10(b), a high frequency band having a large vibration level is eliminated from a graph 901 of the function P(f) of FIG. 10(a) and hence, a graph 902 of the function P′(f) including only a low frequency band having a low vibration level is acquired.

At Step 704, the low vibration MPG pulse computation unit 302 performs inverse Fourier transform on the function P′(f) including only a low frequency band having a low vibration level, and acquires a function p′(t) indicative of a MPG pulse waveform which does not include a large frequency component having a large vibration level. A waveform of an MPG pulse 803 shown in FIG. 9(b) illustrates the function p′(t). The waveform of an MPG pulse 803 shown in FIG. 9(b) has two smooth-shaped peaks 807. Side lobes 806 slightly appear on both sides of the MPG pulse 803.

Then, at next Step 705, the low vibration MPG pulse computation unit 302 performs processing which multiplies the MPG pulse 803 by a window function v(t) in a time region for eliminating the side lobes 806. The window function v(t) is a function different from the abovementioned window function W(f), and is defined by a time 207 within which the MPG pulse 803 is allowed to be applied. The time 207 within which the MPG pulse 803 is allowed to be applied is exactly shown in FIG. 2. In many cases, the application time 207 is determined based on an echo time TE corresponding to an imaging condition. The window function v(t) is generated such that a value of a function p′(t) of the MPG pulse 803 outside the maximum application time 207 within which the MPG pulse 803 is allowed to be applied is set to zero and hence, the function p′(t) is cancelled. For example, as the window function v(t), a sin function expressed by Expression (3) is used.

$$\begin{matrix}[ Expression 3 ] & \\ v ( t ) = sin ( \frac{t \times π}{Width} + π ) & ( 3 )\end{matrix}$$

In the Expression (3), “t” is a time elapsed from a point of time that the MPG pulse 803 is started to be applied. “Width” is an application time 207 allowed to the MPG pulse.

FIG. 9(c) shows an MPG pulse 804 of a function p″(t) which the low vibration MPG pulse computation unit 302 acquires by multiplying the function p′(t) of the MPG pulse 803 by a window function v(t) in Step 705. As shown in FIG. 9(c), the side lobes 806 of the MPG pulse 803 are eliminated from the MPG pulse 804.

Finally, at Step 706, the low vibration MPG pulse computation unit 302 acquires an MPG pulse 805 of a function p′″(t) as shown in FIG. 9(d) by adjusting an amplitude (gradient magnetic field strength) of the MPG pulse 804 such that a b-factor of the MPG pulse 804 becomes equal to a value designated in setting an imaging condition (that is, a value of the MPG pulse 801 shown in FIG. 9(a)). In place of calculating the b-factor, the amplitude (gradient magnetic field strength) may be adjusted such that an area (=gradient magnetic field strength×application time) of the MPG pulse 805 becomes equal to an area of the MPG pulse 804.

In accordance with the steps described above, the MPG pulse 805 of the low vibration is calculated by the low vibration MPG pulse computation unit 302. The low vibration MPG pulse computation unit 302 stores the acquired MPG pulse 805 of the low vibration in the low vibration MPG pulse waveform storage unit 314 of an RM 22. By executing a DWI sequence by replacing a conventional MPG pulse with the obtained MPG pulse 805 having low vibration, it is possible to reduce vibration attributed to an MPG pulse compared to the prior art.

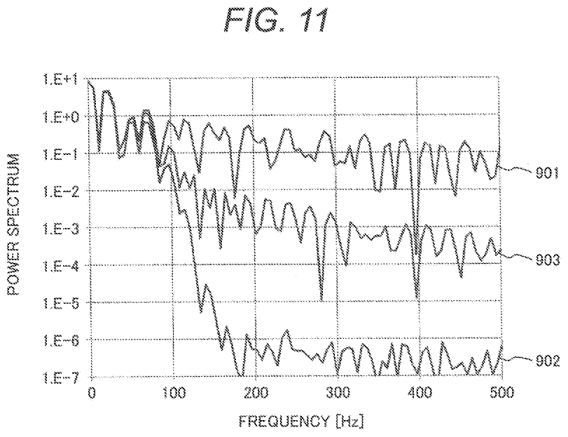

FIG. 10(c) shows a frequency spectrum 903 of the MPG pulse 805 which is a final result. FIG. 11 collectively shows a frequency spectrum 901 of the MPG pulse 802 before high frequency band is removed shown in FIG. 10(a), a frequency spectrum 902 after the high frequency band is removed shown in FIG. 10(b), and a frequency spectrum 903 shown in FIG. 10(c).

A logarithm is taken on an axis of ordinates in FIG. 11. In a frequency spectrum 901 shown in FIG. 11, a sum of power spectrum of equal to or more than maximum frequency Tf(105 Hz) set in Step 402 is approximately 36.5% of a sum of power spectrum of all frequency bands of the frequency spectrum 901. On the other hand, a sum of power spectrum of the frequency spectrum 902 equal to or more than maximum frequency Tf(105 Hz) is merely approximately 0.015% with respect to the sum of the power spectrum of all frequency bands of the frequency spectrum 901. That is, it is understood that the bands of frequency Tf(105 Hz) or more having large vibration levels can be almost eliminated by applying the window function generated in Steps 401 to 404 to the frequency spectrum 901 in Step 703.

On the other hand, a sum of the power spectrum of maximum frequency Tf(105 Hz) or more of the frequency spectrum 903 is approximately 0.45% with respect to a sum of the power spectrum of all frequency bands of the frequency spectrum 901, and is increased compared to the frequency spectrum 902. This is because the side lobes are eliminated using the window function v(t) in Step 705. However, to compare with the frequency spectrum 901 of the MPG pulse 802 before the high frequency band is removed, a sum of the power spectrum of equal to or more than maximum frequency Tf(105 Hz) which is a region where a response gain of vibration is high is reduced from approximately 36.5% to approximately 0.45% so that the sum of the power spectrum is reduced to approximately 1/81 of the initial value. Accordingly, a large vibration reducing effect can be obtained by using the frequency spectrum 903 of the MPG pulse 805 which is the final result.

As has been described heretofore, an MPG pulse which applies a gradient magnetic field having a large strength generates magnetic field vibration and vibration in a subject's position thus giving rise to drawbacks such as lowering of formability of an image or discomfort to a subject. However, in this embodiment, to prevent a phenomenon that force generated in the gradient magnetic field coil when a current is passed through the gradient magnetic field coil is conveyed to the subject by way of a propagation path including the support for the gradient magnetic field coil and the table, and fluctuation is caused in the subject's position, a waveform of the gradient magnetic field pulse is determined so as to reduce a vibration transmissibility through the abovementioned propagation path.

That is, the shape of the MPG pulse is determined by frequency components having low response level while avoiding frequency components having high response level in a vibration frequency characteristic of the MRI device, and the MPG pulse is applied. Accordingly, vibration generated by applying the MPG pulse can be reduced. As a result, it is possible to suppress lowering of formability of an image which occurs when a gradient magnetic field is not applied with an intended strength, the reflection of the motion of a subject due to vibration on a contrast of an image, and discomfort of the subject.

Further, vibration can be reduced without extending an application time of an MPG pulse and hence, vibration can be reduced while minimizing the extension of an echo time (TE).

### Second Embodiment

An MRI device according to the second embodiment is described.

In the first embodiment, the configuration is adopted where one vibration frequency characteristic is preliminarily obtained using the whole gradient magnetic field coil 9 as one vibration source. In the second embodiment, a vibration frequency characteristic is obtained for respective coils on X, Y, and Z axes of a gradient magnetic field coil 9 which is a vibration source.

To be more specific, a vibration frequency characteristic of an MRI device (table 100) is preliminarily obtained for the respective X, Y, and Z axes coils which form the gradient magnetic field coil 9, and the vibration frequency characteristic of the MRI device (table 100) is stored in a vibration frequency characteristic storage unit 311 of an MRI device of a RAM 22.

The configurations and processing of the MRI device according to the second embodiment substantially equal to the corresponding configurations and processing of the MRI device according to the first embodiment are omitted, and only the configurations and processing which differ are described.

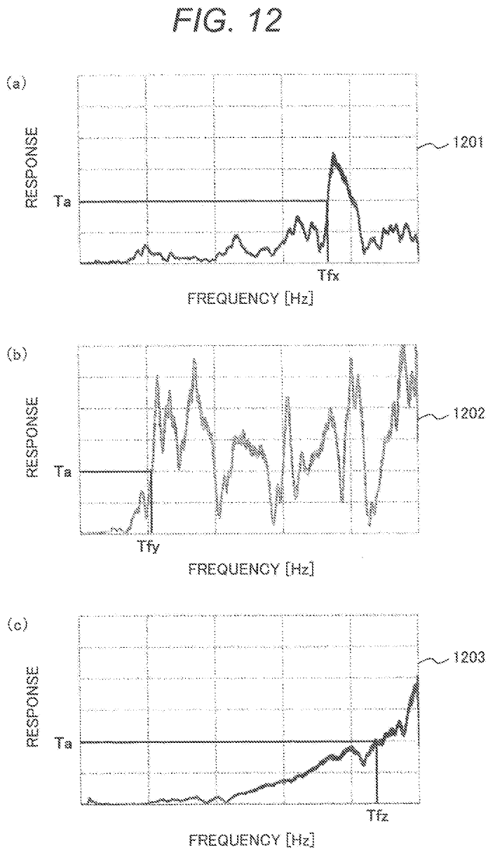

First, although the flow of a window function computation unit 301 is exactly equal to the flowchart shown in FIG. 4 in the same manner as the first embodiment, the second embodiment differs from the first embodiment with respect to the content of the processing. At Step 401, the window function computation unit 301 reads a vibration frequency characteristic of an MRI device (table 100) for respective X, Y, and Z coils (vibration sources) of a gradient magnetic field coil 9 of the MRI device from a vibration frequency characteristic storage unit 311 of a RAM 22. FIG. 12 shows an example of a vibration frequency characteristic of the MRI device (table 100) for respective X, Y, and Z coils (vibration sources) in the second embodiment. In FIG. 12, a graph 1201 indicates the vibration frequency characteristic when the coil on the X axis of the gradient magnetic field coil 9 is driven, a graph 1202 indicates the vibration frequency characteristic when the coil on the Y axis of the gradient magnetic field coil 9 is driven, and a graph 1203 indicates the vibration frequency characteristic when the gradient magnetic field coil on the Z axis is driven. In this embodiment, the vibration frequency characteristics on the X, Y, and Z axes are expressed by Expression (4).

$$\begin{matrix}[ Expression 4 ] & \\ \{ \begin{matrix}VFC ( x , f ) = Max [ ACC ( x , Axis , f ) ] \\ VFC ( y , f ) = Max [ ACC ( y , Axis , f ) ] \\ VFC ( z , f ) = Max [ ACC ( z , Axis , f ) ]\end{matrix} & ( 4 )\end{matrix}$$

In Expression (4), VFC(x, f), VFC(y, f), VFC(z, f) respectively indicate the vibration frequency characteristics when the coils on the X, Y, and Z axes of the gradient magnetic field coil 9 are respectively driven. “f” indicates frequency, Max[ ] is a function indicating the maximum value within [ ]. “ACC( )” is a data row of acceleration measured for each direction of shake source, each direction of acceleration, and each frequency. “Axis” indicates the direction (X, Y, Z) of acceleration.

At processing 402, a window function computation unit 301 sets frequency bands for making an MPG pulse a low vibration pulse for the respective X, Y, and Z axes from the vibration frequency characteristic of the MRI device read at Step 401. In the same manner as the first embodiment, an allowable maximum response gain Ta is determined, and sets the lowest frequencies Tfx, Tfy, Tfz among the frequencies indicating a response gain exceeding a response gain Ta as the maximum frequency constituting an MPG pulse for respective axes X, Y, and Z.

At Step 403, the window function computation unit 301 calculates a window function for generating a low vibration MPG for X, Y, and Z axes respectively using the set maximum frequencies Tfx, Tfy, Tfz. Calculation processing of the window function is performed in the same manner as the first embodiment. The window functions calculated for X, Y, and Z axes respectively are stored in a low vibration MPG window function storage unit 312 of the RAM 22.

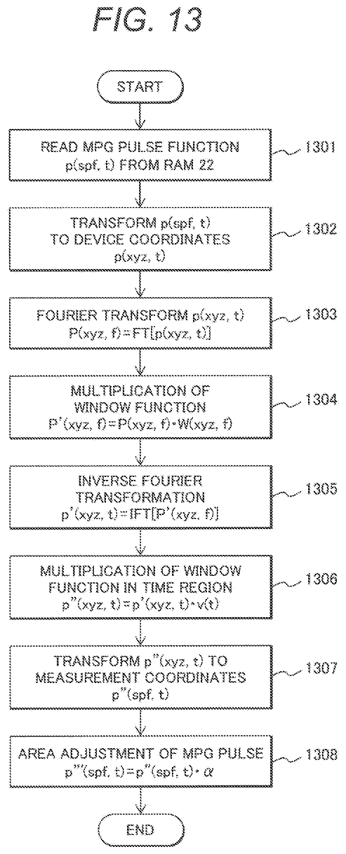

Although the flow of processing of the low vibration MPG pulse computation unit 302 is substantially equal to the corresponding flow in the first embodiment, a point which makes the second embodiment different from the first embodiment is described using the flow shown in FIG. 13. First, at Step 1301, the low vibration MPG pulse computation unit 302 reads a function indicative of an MPG pulse waveform used in a pulse sequence for DWI from an MPG pulse waveform storage unit 313 in the RAM 22. Since the MPG pulse is defined in general by a measurement coordinate system (slice direction (s), phase direction(p), frequency encoding direction (f)), at Step 1301, an MPG pulse to be read is also a function of the measurement coordinate system. At step 1302, functions of an MPG pulse of the measurement coordinate system are converted to functions of a device coordinate system (X axis, Y axis, Z axis) in the same manner as a vibration source in accordance with the following Expression.

$$\begin{matrix}[ Expression 5 ] & \\ [ \begin{matrix}p ( x , t ) \\ p ( y , t ) \\ p ( z , t )\end{matrix} ] = {R}_{OM} [ \begin{matrix}p ( s , t ) \\ p ( p , t ) \\ p ( f , t )\end{matrix} ] & ( 5 )\end{matrix}$$

In Expression (5), R_{OM} indicates an oblique matrix for converting the measurement coordinate system (slice direction, phase direction, frequency encoding direction) to a device coordinate system (X axis, Y axis, Z axis).

Next, at Step 1303, the low vibration MPG pulse computation unit 302 performs Fourier transform on functions p(x, t), p(y, t), p(z, t) of an MPG pulse of the device coordinate system respectively thus acquiring functions P(x, f), P(y, f), P(z, f) respectively indicating frequency spectrum.

At Step 1304, the low vibration MPG pulse computation unit 302 reads a window function W(f) stored at Step 404, multiplies the functions P(x, f), P(y, f), P(z, f) indicative of frequency spectrum by the window function W(f) thus acquiring functions P′(x, f), P′(y, f), P′(z, f) from which high frequency components having large vibration levels are eliminated.

At Step 1305, the low vibration MPG pulse computation unit 302 performs an inverse Fourier transform on the functions P′(x, f), P′(y, f), P′(z, f) from which the high frequency components having the large vibration levels are eliminated thus acquiring functions p′(x, t), p′(y, t), p′(z, t) of the MPG pulse waveform.

At Step 1306, the low vibration MPG pulse computation unit 302 multiplies the functions p′(x, t), p′(y, t), p′(z, t) of the MPG pulse waveform by a window function v(t) in a time region thus acquiring functions p″(x, t), p″(y, t), p″(z, t) of the MPG pulse waveform from which side lobes are eliminated. For example, the previously-described Expression (3) is used as the window function v(t).

At Step 1307, the low vibration MPG pulse computation unit 302 converts the functions p″(x, t), p″ (y, t), p″ (z, t) of the MPG pulse waveform of the device coordinate system to functions p″(s, t), p″(p, t), p″(f, t) of the MPG pulse waveform of the measurement coordinate system in accordance with the following Expression.

$$\begin{matrix}[ Expression 6 ] & \\ [ \begin{matrix}{p}^{n} ( s , t ) \\ {p}^{n} ( p , t ) \\ {p}^{n} ( f , t )\end{matrix} ] = {R}_{OM}^{T} [ \begin{matrix}{p}^{n} ( x , t ) \\ {p}^{n} ( y , t ) \\ {p}^{n} ( z , t )\end{matrix} ] & ( 6 )\end{matrix}$$

In Expression (6), as described previously, Rₒₘ is an oblique matrix for converting a measurement coordinate system (slice direction s, phase direction p, frequency encoding direction f) to a device coordinate system (X axis, Y axis, Z axis). For converting the functions indicative an MPG pulse of the device coordinate system to the measurement coordinate system, an inverse matrix of the oblique matrix is multiplied to the functions indicative of the MPG pulse of the device coordinate system. Since the oblique matrix is a rotation matrix, the inverse matrix of the oblique matrix is substantially equal to a transposed matrix.

Finally, at Step 1308, the low vibration MPG pulse computation unit 302 readjusts amplitude such that b-factors of the function p″(s, t), p″(p, t), p″(f, t) of the MPG pulse waveform of the measurement coordinate system become equal to values designated in setting imaging conditions thus acquiring the function p′″(s, t), p′″ (p, t), p′″ (f, t) of the MPG pulse waveform of the measurement coordinate system.

As has been described above, in the MRI device of the second embodiment, vibration generated by application of a gradient magnetic field pulse can be reduced. Accordingly, there is no possibility that the motion of a subject due to vibration is reflected on a contrast of an image. Further, the unwanted information is not mixed into an echo signal and hence, it is possible to prevent lowering of an image due to vibration. Still further, it is unnecessary to extend an application time of an MPG pulse and hence, vibration can be reduced whereby it is possible to reduce vibration while minimizing the extension of an echo time (TE).

Particularly, in the second embodiment, a vibration frequency characteristic is prepared for respective vibration sources (coils on X axis, Y axis and Z axis of the gradient magnetic field coil 9) and these vibration frequency characteristics are applied to the MPG pulse. Accordingly, it is unnecessary to excessively limit a frequency band of the MPG pulse on the axis along which the vibration is minimally generated. For example, in the example shown in FIG. 12, a response gain on the Y axis (graph 1202) is high so that it is necessary to limit frequency in a wide range compared to other axes. However, a response gain on the Z axis (graph 1203) is low so that a band where frequency is to be limited becomes narrow.

As a result, at Step 1306, by multiplying functions p′ (x, t), p′ (y, t), p′ (z, t) of an MPG pulse waveform by a window function v(t) within a time region thus eliminating side lobes, it is possible to acquire an advantageous effect that the increase of a sum of power spectrum of maximum frequency Tf or more having high vibration levels can be avoided with respect to the Z axis where a band in which frequency is limited is narrow.

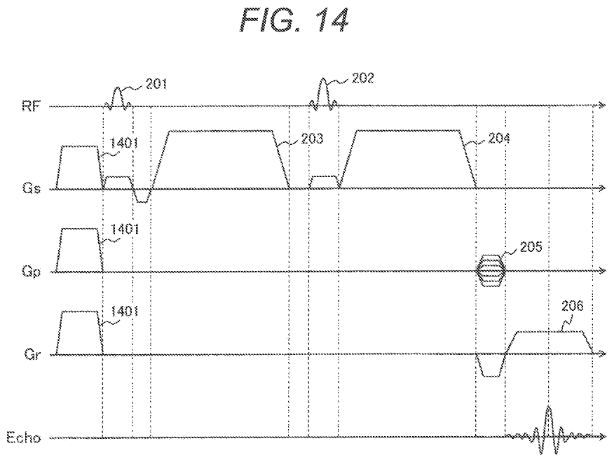

Further, in the first and the second embodiments, the description has been made with respect to the cases where an MPG pulse is applied as' a gradient magnetic field pulse as an example. However, the embodiments of the present invention are not limited to an MPG pulse, and are applicable to other gradient magnetic field pulses. For example, the embodiments of the present invention may be applicable to a gradient magnetic field pulse for extinguishing transverse magnetization of a spin referred to as a crusher. FIG. 14 is an example of a pulse sequence for DWI which is accompanied with the crusher. A gradient magnetic field pulse 1401 in FIG. 14 is the crusher. Although it depends on designing of a pulse sequence, there may be the cases where a gradient magnetic field strength of the crusher is high. In such cases, in the same manner as the MPG pulse, the crusher may cause vibration.

The present invention is not limited to the abovementioned first and the second embodiments, and various modifications can be conceivable. For example, a silencing unit may be further provided for reducing noises. In this case, by supplying currents of various frequencies to the gradient magnetic field coil and by measuring noise levels for respective frequencies by a microphone, frequency characteristics of the noise levels are obtained preliminarily. The silencing unit obtains frequency bands having large noise levels based on the frequency characteristics of the noise levels, and eliminates the frequency bands having the large noise level from frequency spectrum which are obtained by applying Fourier transform to the gradient magnetic field pulse.

In this case, the frequency band having the large noise level differs from the frequency band having the large vibration level of the previously mentioned embodiments and hence, the frequency band having the large noise level may be eliminated from the frequency spectrum of the gradient magnetic field pulse and, thereafter, the frequency band having the large vibration level may be limited by the window functions in the first and the second embodiment as described previously. Alternatively, the frequency band having the large vibration level may be limited and, thereafter, the frequency band having the large noise level may be eliminated. Then, a waveform of the gradient magnetic field pulse is determined by applying inverse Fourier transform to the frequency spectrum of the frequency band after elimination or limitation.

As the previously mentioned functions indicated in Expression (1) and Expression (2), a Gaussian window or a Blackman window can be used besides the previously mentioned functions.

In selecting a frequency component having a low response gain from a vibration frequency characteristic of the MRI device, a high frequency component having a low response gain may be included besides a low frequency component.

### REFERENCE SIGNS LIST

1 . . . subject,
2 . . . static magnetic field generation system,
3 . . . gradient magnetic field generation system,
4 . . . sequencer,
5 . . . transmission system,
6 . . . reception system,
7 . . . signal processing system,
8 . . . digital signal processor,
9 . . . gradient magnetic field coil,
10 . . . gradient magnetic field power supply,
11 . . . high frequency oscillator,
12 . . . modulator,
13 . . . high frequency amplifier,
14a . . . high frequency coil (transmission coil),
14b . . . high frequency coil (reception coil),
15 . . . signal amplifier,
16 . . . quadrature phase detector,
17 . . . A/D converter,
18 . . . magnetic disk,
19 . . . optical disk,
20 . . . display,
21 . . . ROM,
22 . . . RAM,
25 . . . operating unit,
27 . . . static magnetic field generating device,
28 . . . imaging space,
100 . . . table,
201 . . . high frequency magnetic field pulse,
202 . . . high frequency magnetic field pulse,
203 . . . first MPG pulse,
204 . . . second MPG pulse,
205 . . . phase encoding gradient magnetic field pulse (GP),
206 . . . frequency encoding gradient magnetic field pulse (Gf),
209 . . . slice selection gradient magnetic field pulse (Gs),
210 . . . echo signal,
301 . . . window function computation unit,
302 . . . low vibration MPG pulse computation unit,
311 . . . vibration frequency characteristic storage unit,
312 . . . low vibration MPG window function storage unit,
313 . . . MPG pulse waveform storage unit,
314 . . . low vibration MPG pulse waveform storage unit

1. A magnetic resonance imaging device comprising:
a static magnetic field generating device supplying a static magnetic field to an imaging space in which a subject is placed; a table for placing a subject in the imaging space; a gradient magnetic field coil applying a gradient magnetic field pulse to the imaging space; a gradient magnetic field power supply supplying a current of a predetermined waveform to the gradient magnetic field coil to generate the gradient magnetic field pulse; a support supporting the gradient magnetic field coil; and a control unit that controls and causes the gradient magnetic field power supply to apply the gradient magnetic field pulse of a predetermined waveform to the imaging space at predetermined timing and execute a predetermined imaging pulse sequence including the gradient magnetic field pulse,
wherein the control unit includes a waveform determination unit determining a waveform of the gradient magnetic field pulse, and
the waveform determination unit is configured to determine a waveform of the gradient magnetic field pulse so as to reduce a vibration transmissibility of a propagation path including the support for the gradient magnetic field coil and the table so as to prevent a force generated in the gradient magnetic field coil when a current is passed through the gradient magnetic field coil from being conveyed to the subject by way of the propagation path and fluctuation is caused in the subject's position.

2. The magnetic resonance imaging device according to claim 1, wherein the waveform determination unit uses a relation between the frequency of a current supplied to the gradient magnetic field coil and magnitude of the resultant vibration in the table, obtained in advance, to select a frequency component with which the magnitude of vibration at the frequency is equal to or lower than a predetermined value, and the waveform of the gradient magnetic field pulse is determined with the selected frequency component.

3. The magnetic resonance imaging device according to claim 1, wherein the waveform determination unit is configured to adjust the waveform such that maximum strength of the waveform of the gradient magnetic field pulse approximately agrees with maximum strength which is capable of being generated by the gradient magnetic field pulse.

4. The magnetic resonance imaging device according to claim 2, wherein the vibration strength is acceleration of the vibration.

5. The magnetic resonance imaging device according to claim 2, wherein the waveform determination unit converts gradient magnetic field pulse of a predetermined waveform used in the imaging pulse sequence to a waveform where the strength of the gradient magnetic field pulse is equal to maximum strength which the gradient magnetic field coil is capable of irradiating, and a product of the strength and an application time agrees with the gradient magnetic field pulse of the predetermined waveform, and selects the frequency component with respect to the gradient magnetic field pulse waveform of the waveform after the conversion.

6. The magnetic resonance imaging device according to claim 2, wherein the waveform determination unit obtains a frequency spectrum by applying a frequency analysis to a gradient magnetic field pulse of a predetermined waveform used in the imaging pulse sequence, selects a frequency band from the frequency spectrum where the vibration strength becomes equal to or lower than a predetermined value, and determines a waveform of the gradient magnetic field pulse by converting the frequency spectrum in the selected frequency band to applied strength distribution in a time axis direction.

7. The magnetic resonance imaging device according to claim 6,
wherein the waveform determination unit includes: a window function computation unit; and a low vibration gradient magnetic field pulse computation unit, and
the window function computation unit generates a window function for selecting a frequency band where the vibration strength becomes equal to or lower than a predetermined value from the frequency spectrum, and
the low vibration gradient magnetic field pulse computation unit adjusts maximum strength of an inclined magnetic field pulse having the predetermined waveform to maximum strength which the inclined magnetic field coil is applicable and, thereafter, performs Fourier transform to obtain the frequency spectrum, selects the frequency band by applying the window function to the obtained frequency spectrum, and performs inverse Fourier transform on a frequency spectrum of the selected frequency band thus determining a waveform of the gradient magnetic field pulse.

8. The magnetic resonance imaging device according to claim 7,
wherein the gradient magnetic field pulse is an MPG pulse used in imaging a diffusion weighted image, and
the low vibration gradient magnetic field pulse computation unit adjusts a waveform such that a b-factor of the gradient magnetic field pulse of the determined waveform agrees with a gradient magnetic field pulse of the predetermined waveform.

9. The magnetic resonance imaging device according to claim 7, wherein the low vibration gradient magnetic field pulse computation unit performs processing for eliminating side lobes of the gradient magnetic field pulse of the determined waveform.

10. The magnetic resonance imaging device according to claim 7, further comprising a reception unit for receiving the selection of a degree of reduction of vibration from a user,
wherein the window function computation unit generates the window function of the frequency band which is different in response to the degree of reduction of vibration which the reception unit receives.

11. The magnetic resonance imaging device according to claim 2,
wherein the gradient magnetic field coil includes a plurality of coils having different directions along which a gradient magnetic field is applied, and a relation between a frequency of a current supplied to the gradient magnetic field coil and a vibration strength of the static magnetic field generating device generated by the frequency is prepared for each of the plurality of coils, and
the waveform determination unit determines a waveform of the gradient magnetic field pulse using the corresponding relation in response to the direction of the gradient magnetic field pulse which the gradient magnetic field coil applies.

12. The magnetic resonance imaging device according to claim 6,
wherein the control unit further includes a silencing unit for eliminating a frequency component of equal to or more than a predetermined value of human audible sensitivity from the predetermined gradient magnetic field pulse used in the imaging pulse sequence, and
the waveform determination unit selects a frequency component whose vibration strength becomes equal to or lower than a predetermined value from a frequency component of the gradient magnetic field pulse after processing performed by the silencing unit.
